(19) United States
(12) Patent Application Publication
(10) Pub. No.: US 2007/0032553 A1
(43) Pub. Date: Feb. 8, 2007

(54) Use of gabaa receptor agonists for the treatment of hearing, vestibular and attention disorders, intention tremor and restless leg syndrome

(76) Inventors: Ruth McKernan, Canterbury (GB); Keith Alan Wafford, Much Hadham (GB)

Correspondence Address: MERCK AND CO., INC, P O BOX 2000, RAHWAY, NJ, 07065-0907, US

(21) Appl. No.: 10/571,656

(22) PCT Filed: Sep. 7, 2004
(86) PCT No.: PCT/GB04/03828

§ 371 Date: Mar. 10, 2006

(30) Foreign Application Priority Data
Sep. 10, 2003 (GB) 0321234.7
Sep. 10, 2003 (GB) 0321236.2
Sep. 10, 2003 (GB) 0321235.4
Sep. 16, 2003 (GB) 0321649.6
Sep. 16, 2003 (GB) 0321650.4
Sep. 19, 2003 (GB) 0322000.1

(51) Int. Cl. A61K 31/195

(52) U.S. Cl. 514/561

(57) ABSTRACT

The invention provides a method of treating a condition selected from hearing disorders, vestibular disorders, attention disorders, intention tremor and restless leg syndrome comprising administering to a human patient in need thereof a therapeutically effective amount of a compound which is an agonist at GABA_{A} receptors which comprise an α 4 subunit and a δ subunit.

This invention relates to the methods of treatment of the human body and to compositions suitable for use therein. In particular, it provides methods and materials for treating hearing disorders (especially tinnitus), vestibular disorders, attention disorders (especially ADHD), intention tremor, and restless leg syndrome.

Tinnitus is the perception of sound in one or both ears or in the head in the absence of an acoustic stimulus. It affects a significant proportion of the population (eg. an estimated 50 million sufferers in the USA alone), but there is currently no specific medical or surgical therapy for tinnitus.

Vestibular disorders are disorders of the inner ear affecting balance, and include Meniere's disease, benign paraxsysmal positional vertigo (BPPV), endolymphatic hydrops and mal de debarquement syndrome. Vestibular disorders (especially Meniere's disease) frequently occur in conjunction with hearing disorders such as tinnitus.

Attention-deficit/hyperactivity disorder (ADHD) is a condition characterised by inattentive, impulsive hyperactive behaviour, and affects some 6% of school age boys in the USA. Although primarily affecting children, in some cases the symptoms persist into adulthood. Several recent studies have implicated the dopamine D₄ receptor in the etiology of ADHD (see, for example, Zhang et al., Neuropsychopharmacology, 2001, 25, 624-632, and references therein).

Intention tremor (also known as action tremor, volitional tremor or kinetic tremor) is an involuntary oscillation of a limb when approaching a target.

Restless leg syndrome (RLS) is a neurological disorder characterised by unpleasant sensations in the legs and an uncontrollable urge to move when at rest in an attempt to relieve these sensations. Sufferers have difficult in sleeping, leading to daytime fatigue, exhaustion, and severe disruption of activities of daily living.

GABA (γ-Aminobutyric acid) is the major inhibitory neurotransmitter in the mammalian central nervous system. Its primary action is through the GABA_{A} receptor, which is composed of a family of functionally diverse subunits that assemble into a pentameric structure. To date there are 17 different subunits identified (α₁₋₆, β₁₋₃, γ₁₋₃, ρ₁₋₂, δ, ε, θ). These subunits have discrete locations with the brain, but the most abundant receptor subtypes have been found to express α, β and γ subunits. The GABA_{A} receptor can be modulated by a number of therapeutic agents, including benzodiazepines, barbiturates, anaesthetics, ethanol and neuroactive steroids. The extent of this modulation is subunit specific. Recombinant studies have shown the α and γ subunits are responsible for benzodiazepine and zinc sensitivity, and β subunits control loreclezole and etomidate sensitivity. α₄ subunits comprise only a small percentage of neuronal subunits, concentrated in hippocampus, striatum, cerebral cortex, thalamus, and basal ganglia. They assemble with β_{2/3} and γ₂ subunits in most areas of the brain but also with β_{2/3} and δ subunits in olfactory bulb, dentate gyrus, and thalamus. Of the 20-27% of thalamic GABA_{A} receptors that contain α₄ subunits, approximately one-third contain γ₂ subunits, and two-thirds contain δ subunits. Compared with other GABA_{A} receptors, those containing α₄ subunits differ in their rectification properties, affinity for GABA, and modulation by benzodiazepine. Receptors containing α₄ and δ subunits lack benzodiazepine binding sites entirely, and those containing α₄, β and γ₂ subunits have a benzodiazepine binding site that is atypical.

Transient expression of ternary GABA_{A} receptors containing the δ subunit is described in Wohlfarth et al, J. Neuroscience, 22, 1541-9 (2002) and Belelli et al, Neuropharmacology, 43, 651-61 (2002), and stable expression of the α₄β₃δ receptor is described in Adkins et al, J. Biol. Chem., 276, 38934-9 (2001) and Brown et al, British J. Pharmacol., 136, 965-74 (2002). According to these papers, such receptors are likely to be involved in epilepsy, drug withdrawal and conditions associated with neurosteroid depletion, especially premenstrual syndrome.

WO 03/063845 discloes the use of ligands for the α2δ receptor (e.g. gabapentin) for treatment of tinnitus.

Bauer and Brozoski, J. Assoc. Res. Otolaryngol., 2, 54-64 (2001) describe an animal model for testing the efficiency of prospective tinnitus therapies.

Gaboxadol (4,5,6,7-tetrahydroisoxazolo[5,4-c]pyridin-3-ol, also known as THIP) is reported (Brown et al, supra) to be a potent agonist at the α4β3δ receptor. Previously, it had been claimed for use in treatment of sleep disorders (WO 97/02813), and subsequently it has been reported to be useful in the treatment of premenstrual syndrome (WO 02/40009; Gulinello et al, NeuroReport, 14, 43-6 (2003)). There are no reports in the literature of the utility of gaboxadol or any other agonist of the α4β3δ receptor in treatment of any of the disease states relevant to the present invention.

In one aspect the invention is the use of a compound which is an agonist at GABA_{A} receptors which comprise an α4 subunit and a δ subunit for the manufacture of a medicament for treatment of a condition selected from hearing disorders, vestibular disorders, attention disorders, intention tremor and restless leg syndrome.

In a second aspect the invention is a method of treating a condition selected from hearing disorders, vestibular disorders, attention disorders, intention tremor and restless leg syndrome comprising administering to a human patient in need thereof a therapeutically effective amount of a compound which is an agonist at GABA_{A} receptors which comprise an α4 subunit and a δ subunit.

In a preferred embodiment, the compound is an agonist at a GABA_{A} receptor which additionally comprises a β subunit, such as the β3 subunit.

Preferably, the GABA_{A} agonist is selective for the receptor comprising an α4 subunit and a δ subunit, and in particular is selective for the α4β3δ receptor over the α4β3γ receptor.

Examples of hearing disorders susceptible to treatment in accordance with the invention include tinnitus, age-related hearing loss, presbycusis and hyperacusis, and in particular tinnitus.

Examples of vestibular disorders susceptible to treatment in accordance with the invention include Meniere's disease, benign paroxsysmal positional vertigo (BPPV), endolymphatic hydrops and mal de debarquement syndrome.

Examples of attention disorders susceptible to treatment in accordance with the invention include ADHD.

GABA_{A} agonists suitable for use in the invention can be identified using cells which express the relevant receptor, in particular cells which stably express the receptor, such as mouse L(-tk) cells engineered to express the α4β3δ receptor as described by Brown et al (supra) and Adkins et al (supra).

Therefore, in accordance with a further aspect, the invention provides the use of cells stably expressing the α4β3δ receptor for identifying compounds suitable for treatment of a condition selected from hearing disorders, vestibular disorders, attention disorders, intention tremor and restless leg syndrome.

In one suitable method for identifying such compounds, GABA-induced membrane potentials in the cells are measured in the presence and absence of putative agonists by fluorescence resonance energy transfer techniques, as described by Adkins et al (supra). In another suitable method, GABA-gated currents are measured in the presence and absence of putative agonists, e.g by patch clamp techniques as described by Brown et al (supra). Typically, the current or potential is measured in the presence of GABA at a concentration lower than that required to elicit the maximum GABA-induced response, e.g. 20% of that concentration. This current or potential represents a baseline signal, and elevation of this signal in the presence of a test compound indicates that the compound in question is an agonist. The magnitude of the elevated signal (expressed as a percentage of the maximum GABA response) is a measure of potency. Compounds which elicit at least 50% of the maximum GABA response are preferred, and compounds which elicit 100% or more of the maximum GABA response are particularly preferred.

A preferred compound for use in the invention is gaboxadol, or a pharmaceutically acceptable salt thereof, such as a pharmaceutically acceptable acid addition salt such as the hydrochloride or hydrobromide.

For use in the invention, the relevant GABA_{A} agonist is formulated as a pharmaceutical composition comprising the active species and a pharmaceutically acceptable carrier. Preferably such compositions are in a unit dosage form such as tablets, pills, capsules, powders, granules, sterile parenteral solutions or suspensions, metered aerosol or liquid sprays, drops, ampoules, transdermal patches, auto-injector devices or suppositories; for oral, parenteral, intranasal, sublingual or rectal administration, or for administration by inhalation or insufflation. The principal active ingredient typically is mixed with a pharmaceutical carrier, e.g. conventional tableting ingredients such as corn starch, lactose, sucrose, sorbitol, talc, stearic acid, magnesium stearate and dicalcium phosphate, or gums, dispersing agents, suspending agents or surfactants such as sorbitan monooleate and poly(ethylene glycol), and other pharmaceutical diluents, e.g. water, to form a homogeneous preformulation composition containing one or both active species, or pharmaceutically acceptable salts thereof When referring to these preformulation compositions as homogeneous, it is meant that the active species is or are dispersed evenly throughout the composition so that the composition may be readily subdivided into equally effective unit dosage forms such as tablets, pills and capsules. This preformulation composition is then subdivided into unit dosage forms of the type described above, generally containing from 0.01 to about 500 mg of the active species. Typical unit dosage forms contain from 0.05 to 100 mg, for example 0.05, 0.1, 0.5, 1, 2, 5, 10, 25, 50 or 100 mg, of the active species. Tablets or pills of the pharmaceutical composition(s) can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the pharmaceutical compositions useful in the present invention may be incorporated for administration orally or by injection include aqueous solutions, liquid- or gel-filled capsules, suitably flavoured syrups, aqueous or oil suspensions, and flavoured emulsions with edible oils such as cottonseed oil, sesame oil or coconut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, poly(ethylene glycol), poly(vinylpyrrolidone) and gelatin.

Pharmaceutical compositions suitable for oral administration are preferred, in particular solid unit dosage forms, preferably tablets or capsules. Suitable formulations and techniques for manufacturing tablets containing gaboxadol and acid addition salts thereof are disclosed in WO 01/22941 and WO 02/094225.

For the treatment of hearing disorders, vestibular disorders, attention disorders, intention tremor and restless leg syndrome, the relevant GABA_{A} agonist is preferably administered at a dose known or estimated to provide occupancy of the intended GABA_{A} receptor. Such dosage levels may be determined by standard methods known to those skilled in the art. The frequency of dosing of the relevant compound (e.g. once, twice, three times or four times per day) may be selected according to the pharmacokinetic profile of the compound concerned. In the case of the preferred compound, gaboxadol, suitable dosage is in the range 0.05 to 1.0 mg/Kg per day, typically 0.1 to 0.5 mg/Kg per day.

In a preferred embodiment of the invention, gaboxadol or a pharmaceutically acceptable salt thereof is administered as a once a day oral dose equivalent to 15 mg or 20 mg of gaboxadol itself to a patient suffering from tinnitus.

In another preferred embodiment, this invention provides the use of gaboxadol or a pharmaceutically acceptable salt thereof in the manufacture of an orally-administrable medicament for the treatment of hearing disorders, in particular tinnitus.

In another preferred embodiment, this invention provides the use of gaboxadol or a pharmaceutically acceptable salt thereof in the manufacture of an orally-administrable medicament for the treatment of vestibular disorders, in particular Meniere's disease or BPPV.

In another preferred embodiment, this invention provides the use of gaboxadol or a pharmaceutically acceptable salt thereof in the manufacture of an orally-administrable medicament for the treatment of attention disorders, especially ADHD.

In another preferred embodiment, this invention provides the use of gaboxadol or a pharmaceutically acceptable salt thereof in the manufacture of an orally-administrable medicament for the treatment of intention tremor.

In another preferred embodiment, this invention provides the use of gaboxadol or a pharmaceutically acceptable salt thereof in the manufacture of an orally-administrable medicament for the treatment of restless leg syndrome.

In another preferred embodiment, this invention provides a method of treating a hearing disorder, especially tinnitus, which comprises the oral administration of gaboxadol or a pharmaceutically acceptable salt thereof to a human patient in need thereof.

In another preferred embodiment this invention provides a method of treating a vestibular disorder, especially Meniere's disease or BPPV, which comprises the oral administration of gaboxadol or a pharmaceutically acceptable salt thereof to a human patient in need thereof.

In another preferred embodiment, this invention provides a method of treating attention disorder, especially ADHD, which comprises the oral administration of gaboxadol or a pharmaceutically acceptable salt thereof to a human patient in need thereof.

In another preferred embodiment, this invention provides a method of treating intention tremor, which comprises the oral administration of gaboxadol or a pharmaceutically acceptable salt thereof to a human patient in need thereof.

In another preferred embodiment, this invention provides a method of treating restless leg syndrome, which comprises the oral administration of gaboxadol or a pharmaceutically acceptable salt thereof to a human patient in need thereof.

In the particularly preferred embodiments listed above the dose is typically in the range 5 to 50 mg, more suitably 10 to 40 mg, and preferably 15 or 20 mg per day of gaboxadol or the equivalent dose of a pharmaceutically acceptable salt thereof, such as the hydrochloride.

### EXAMPLES

In the following examples gaboxadol or an equivalent amount of its hydrochloride salt is formulated in tablet form by conventional methods or by the methods disclosed in WO 01/22941 or WO 02/094225.

### Example 1

### Treatment of Hearing Disorders

A 15 mg or 20 mg dose of gaboxadol or the equivalent amount of the hydrochloride salt in tablet form is administered to a patient suffering from a hearing disorder, in particular tinnitus. The dose is repeated daily until normal hearing is restored.

### Example 2

### Treatment of Vestibular Disorders

A 15 mg or 20 mg dose of gaboxadol or the equivalent amount of the hydrochloride salt in tablet form is administered to a patient suffering from a vestibular disorder, in particular Meniere's disease or BPPV. The dose is repeated daily until the symptoms subside.

### Example 3

A dose of gaboxadol hydrochloride equivalent to 15 mg of 20 mg of gaboxadol in tablet form is administered to a patient suffering from tinnitus.

### Example 4

### Treatment of ADHD

A 15 mg dose of gaboxadol or the equivalent amount of the hydrochloride salt in tablet form is administered to an adolescent male patient suffering from ADHD. The dose is repeated daily until normal behaviour is restored.

### Example 5

### Treatment of ADHD

A 20 mg dose of gaboxadol or the equivalent amount of the hydrochloride salt in tablet form is administered to an adult male suffering from ADHD. The dose is repeated daily until the symptoms subside.

### Example 6

### Treatment of Intention Tremor

A 15 mg dose of gaboxadol or the equivalent amount of the hydrochloride salt in tablet form is administered to a patient suffering from intention tremor. The dose is repeated daily until the symptoms subside.

### Example 7

### Treatment of Intention Tremor

A 20 mg dose of gaboxadol or the equivalent amount of the hydrochloride salt in tablet form is administered to a patient suffering from intention tremor. The dose is repeated daily until the symptoms subside.

### Example 8

### Treatment of Restless Leg Syndrome

A 15 mg dose of gaboxadol or the equivalent amount of the hydrochloride salt in tablet form is administered to a patient suffering from restless leg syndrome. The dose is repeated daily until the symptoms subside.

### Example 9

### Treatment of Restless Leg Syndrome

A 20 mg dose of gaboxadol or the equivalent amount of the hydrochloride salt in tablet form is administered to a patient suffering from restless leg syndrome. The dose is repeated daily until the symptoms subside.

1-10. (canceled)

11. A method of treating a condition selected from a hearing disorder, a vestibular disorder, an attention disorder, intention tremor and restless leg syndrome comprising administering to a human patient in need thereof a therapeutically effective amount of a compound which is an agonist at GABA_{A} receptors which comprise an α4 subunit and a δ subunit or a pharmaceutically acceptable salt thereof.

12. The method of claim 11 wherein the condition is tinnitus.

13. The method of claim 11 wherein the condition is a vestibular disorder.

14. The method of claim 13 wherein the vestibular disorder is Meniere's disease or benign paraxsysmal positional vertigo.

15. The method of claim 11 wherein the condition is an attention disorder.

16. The method of claim 15 wherein the attention disorder is attention deficit hyperactivity disorder.

17. The method of claim 11 wherein the condition is intention tremor.

18. The method of claim 11 wherein the condition is restless leg syndrome.

19. The method of claim 11 wherein the compound is an agonist at a GABA_{A} receptor which additionally comprises a β subunit.

20. The method of claim 11 wherein the compound is a GABA_{A} agonst which is selective for the α4β3δ receptor over the α4β3γ receptor.

21. The method of claim 11 wherein the compound is gaboxadol or a pharmaceutically acceptable salt thereof.

22. A method of treating a condition selected from hearing disorders, vestibular disorders, attention disorders, intention tremor and restless leg syndrome comprising administering to a human patient in need thereof a therapeutically effective amount of gabaxadol or a pharmaceutically acceptable salt thereof.

23. The method of claim 22 wherein gabaxadol or a pharmaceutically acceptable salt thereof is adminstered as a once a day oral dose equivalent to 15 mg or 20 mg of gaboxadol.

24. The method of claim 22 wherein the condition is tinnitus.

25. The method of claim 22 wherein the condition is a vestibular disorder.

26. The method of claim 25 wherein the condition is Meniere's disease or benign paraxsysmal positional vertigo.

27. The method of claim 22 wherein the condition is an attention disorder.

28. The method of claim 27 wherein the condition is attention deficit hyperactivity disorder.

29. The method of claim 22 wherein the condition is intention tremor.

30. The method of claim 22 wherein the condition is restless leg syndrome.
